(19) United States
(12) Patent Application Publication
(10) Pub. No.: US 2018/0093032 A1
(43) Pub. Date: Apr. 5, 2018

(54) Targeted apheresis using binding agents or ligands immobilized on membranes

(71) Applicant: Henry John Smith, Temecula, CA (US)

(72) Inventor: Henry John Smith, Temecula, CA (US)

(21) Appl. No.: 15/731,757

(22) Filed: Jul. 31, 2017

Related U.S. Application Data
(60) Provisional application No. 62/494,584, filed on Aug. 15, 2016.

(51) Int. Cl. A61M 1/36; A61K 35/14

(52) U.S. Cl. CPC A61M 1/362; A61M 1/3693; A61K 35/14

(57) ABSTRACT

This invention teaches a method of targeted apheresis that can be used to treat a variety of different diseases including infectious diseases, autoimmune disorders, and reducing circulating biomarkers associated with certain disorders e.g. pre-eclampsia. This invention discloses the use of apheresis membranes as the support matrix upon which one or more different binding agents (e.g. antibodies, aptamers, binding peptides, soluble receptors), and/or ligands (e.g. antigens, serum proteins, hormones, cytokines and cell markers) are attached. During targeted apheresis the patient's blood will come into contact with one or more of these immobilized binding agents or ligands and any targeted harmful factor in the blood will be bound out of circulation. The cleaned blood is then returned to the patient. Reducing the concentration of the harmful substance in blood will alleviate the symptoms of the disease.

### CROSS-REFERENCE TO RELATED APPLICATIONS

This utility patent application claims priority to Provisional Patent Application Ser. No. 62/494,584 filed Aug. 15, 2016 entitled TARGETED APHERESIS USING APHERESIS MEMBRANES.

### STATEMENT RE: FEDERALLY SPONSORED RESEARCH/DEVELOPMENT

Not Applicable

### BACKGROUND INFORMATION

Apheresis is a procedure to remove harmful substances present in the blood of a patient by processing the blood outside the body thru an apheresis device that removes the harmful substance and returns the cleaned blood back into the patient. Typically this is done using a semi-permeable porous membrane capable of separating out the harmful substance based on its molecular weight and/or physical size. By selecting a membrane with a uniform pore size that is larger than the size of the substance to be removed and smaller than the size of normal blood components it is possible to filter out or dialyze out the offending substance and still retain the normal blood constituents. The following examples illustrate the different apheresis methods used to treat various diseases:

Hemodialysis in which the blood of a patient with renal failure is dialyzed against water or a physiological solution so that toxins in the blood can diffuse thru the semi-permeable dialysis membrane and be removed.
Hemofiltration in which the blood of a patient with fluid overload (e.g. congestive heart failure) is filtered under pressure thru a semi-permeable membrane so that the excess water is filtered out and can be removed.
Plasmapheresis in which the blood of a patient is separated into the plasma fraction and the cellular fraction (i.e. red blood cells, white blood cells and platelets) using centrifugation or by filtration thru a porous membrane. Typically the plasma fraction containing the harmful substance is discarded and replaced with fresh plasma administered to the patient.
Therapeutic apheresis in which the blood is separated into the plasma fraction and the cellular fraction. The plasma fraction is then treated using an absorption column to remove harmful substances before being remixed with the cellular fraction and returned to the patient (e.g. a charcoal filled device to absorb out small molecules such as drugs, the Prosorba® column to remove the Immunoglobulin fraction including autoantibodies and immune complexes; and the Liposorber® column to remove Low Density Lipoprotein).
Targeted apheresis is a more specific method of therapeutic apheresis in which blood or plasma containing a harmful substance is allowed to react with an immobilized binding agent or ligand (e.g. an antibody or an antigen) that will specifically bind out that particular harmful substance without affecting normal blood proteins. The cleaned blood or plasma is then returned to the patient.

The novelty of this invention is that it teaches a method of targeted apheresis that is not disclosed, anticipated or suggested in the prior art. For example, there are numerous publications in the prior art describing therapeutic apheresis utilizing a variety of binding agents immobilized on different types of beads and there are several apheresis devices that have been commercialized. The Prosorba® apheresis column device utilizes Protein A immobilized on silica beads to remove immunoglobulins and immune complexes as a means of treating rheumatoid arthritis and other autoimmune disorders. The Immunosorba® apheresis column device utilizes anti-Immunoglobulin antibodies immobilized on agarose beads to remove Immunoglobulins to treat autoimmune diseases, This conventional method of therapeutic apheresis typically separates out the plasma fraction and then processes the plasma thru a column of beads coated with the binding agent or ligand. The procedure is commonly referred to as “immunoadsorption”.

There is however, no teaching or suggestion in the prior art of utilizing binding agents and/or ligands immobilized on membranes within an apheresis device as a means of removing harmful substances directly from whole blood using targeted apheresis.

### SUMMARY

This invention teaches a method of targeted apheresis that utilizes membranes as the support matrix upon which a variety of binding agents (e.g. antibodies, aptamers, peptides, soluble receptors), and/or a variety of ligands (e.g. antigens, serum proteins), are immobilized. During apheresis the patient's blood will come into contact with the immobilized binding agent or ligand, whereupon the harmful substance in the blood will bind to it and be removed, leaving the cleaned blood to be returned to the patient.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a side view of one embodiment of a targeted apheresis device of the present disclosure.

FIG. 2a is a front view of another embodiment of the present disclosure having a pleated membrane.

FIG. 2b is a side view of the embodiment shown in FIG. 2a.

FIG. 3a is a front view of another embodiment of the present disclosure having multiple membrane sheets arranged in parallel.

FIG. 3b is a side view of the embodiment shown in FIG. 3a.

FIG. 4 is a top view of another embodiment of the present disclosure having multiple membrane sheets arranged in a radial fashion.

### DETAILED DESCRIPTION

There are a wide variety of membranes that can be utilized in this invention. They will vary in their chemical composition and porosity depending on their purpose. The membrane may be composed of fibers or woven into a mesh; or manufactured as a sheet of porous material. The main attribute of the membrane is that the pores or mesh provide a large internal surface area for attaching the binding agent. In the preferred embodiment of this invention the membrane is the type of membrane used in apheresis. However in the instant invention it is important to note that the membrane is not primarily utilized for its capacity for filtration but is instead used because of its large internal surface area to which binding agents can be attached. In this context it should be noted that while the typical apheresis membrane has pores that are too small to permit entry of red blood cells and other cellular elements; the plasma fraction of the blood is able to penetrate into the pores where any harmful factor present in the plasma will be bound out by the binding agent attached to the internal surface of the pores. In this invention the term membrane will include semi-permeable membranes, permeable membranes and in certain instances it will also include membranes that are essentially impermeable.

There are a variety of membranes manufactured with different chemical and physical properties. For example, they are manufactured from a variety of materials including but not limited to: cellulose diacetate (Plasma-APOS); polypropylene (Fenwal CPS-10); polysulfone (Sulflux-FS); polymethylmethacrylate (Plasmax-PS05). They will vary in their density, membrane thickness, surface charge, and pore size. The arrangement of the membranes can be in the form of flat sheets or pleated sheets or as stacked microtubules enclosed within a chamber. In the preferred embodiment of this invention the membranes commonly used in conventional apheresis and their arrangement within a typical apheresis cartridge device are used to provide the support matrix upon which the binding agents or ligands are immobilized.

The primary purpose of utilizing stacked microtubules as the support matrix in targeted apheresis is to provide the maximum surface area for attachment of the binding agent. When harmful factors in the blood come into contact with the binding agent they are bound out and the cleaned blood returned to the patient. In this respect the membrane surface area reported by manufactures of apheresis cartridges typically range from 0.2 m2 to 2.0 m2. However, it is important to note that the surface area of the membrane upon which the binding agent or ligand is immobilized is actually much larger because of the presence of numerous pores in the membrane. The internal surface area of these pores provides for a very large surface area for attachment of the binding agent in addition to the outer surface area of the membrane. Although the pore size of the membrane is too small to permit entry of red blood cells into the membrane itself the plasma component in whole blood can flow through the pores of the membrane and harmful substances present can be bound out by the immobilized binding agent or ligand.

The novelty of this invention is the teaching of using an apheresis membrane as a support matrix that provides a large surface area upon which various binding agents or ligands can be immobilized. When whole blood or plasma comes into contact with the membrane the plasma fraction will permeate thru the membrane and in doing so will be exposed to the binding agent or ligand coating the outer surfaces of the membrane, and also coating the internal surface of the pores. This will maximize the interaction of any undesirable substance in the plasma with the immobilized binding agent or ligand and its efficient removal from circulation.

There are a variety of methods for attaching the binding agent or ligand to the support matrix. One method is by direct adsorption to the membrane. Typically, the binding agent or ligand is dissolved in water or a coating buffer and used to fill the membrane in the apheresis device. After a period of time to allow the material to adsorb onto the membrane the device is flushed with distilled water or buffer to remove unbound material and then stored in a preservative solution; or the device is emptied and stored dry at room temperature or at 4 C. To maintain stability the device may be vacuum packed or stored under nitrogen.

Another method is covalent binding of the binding agent or ligand to the support matrix. On surfaces that are aminated or carboxylated, covalent coupling is achieved using bifunctional crosslinkers that couple the amine or carboxyl group on the surface to a functional group, such as an amine or sulfhydryl, on the biomolecule. Selection of the crosslinker will determine the type of covalent bond that will be formed. Functional and covalently reactive groups, such as N-oxysuccinimide, maleimide and hydrazide groups, can also be grafted onto a suitable surface support via a photolinkable spacer arm resulting in a stable, yet reactive surface to covalently attach the binding agent or ligand.

Another method that can be used is to pre-treat the membrane with 3-aminopropyltriethoxysilane (APTES) and to cross-link the binding agent or ligand to the APTES functionalized surface using 1-Ethyl-3-(3-dimethylaminopropyl) carbodiimide (EDC). In one modification of this method the binding agent is mixed with APTES and applied to the membrane and incubated to allow covalent binding to occur.

Another method is to use a linking agent to attach the binding agent or ligand to the membrane. For example, avidin is covalently attached to the membrane while the binding agent or ligand in solution is biotinylated. When the biotinylated binding agent or ligand comes into contact with the avidin it will bind to it and in turn will thus become attached to the membrane surface. The avidin/biotin system has the advantage of increasing the concentration of immobilized binding agent or ligand on the membrane because each avidin molecule is capable of binding to four biotin molecules. It also orients the binding agent or ligand so that the active site on the molecule is exposed.

After the binding agent or ligand is attached to the membrane any remaining active surfaces can be blocked using a solution of human albumin or similar blocking material.

Or the blocking step can be performed at the time of apheresis by priming the device with a solution of human albumin or similar blocking material. In some instances the membrane is blocked with an anti-coagulant such as heparin in order to prevent clot formation within the device.

There are numerous established methods of attaching proteins, aptamers, and peptides to a variety of surfaces. For example the book “Bioconjugation” (Academic Press 2013) describes the different methods of attaching various biomolecules to a variety of support matrixes. Said materials and methods by reference are therefore included within the scope of this invention. Any particular method selected will depend on the moiety to be attached and the chemical composition of the support matrix. These and other means of attachment will be obvious to one of skill in the art and are therefore considered to be within the spirit and scope of this invention.

Also, there are a variety of methods of storage of the targeted apheresis device depending on the stability of the attached moiety. Preferably, if the binding agent or ligand is stable the device can be stored dry in a vacuum pack at room temperature or at 4 C. Alternatively, it can be filled with a preservative solution and stored at 4 C. These and other means of preservation and storage of the apheresis device will be obvious to one of skill in the art and are therefore considered to be within the spirit and scope of this invention.

The procedure for targeted apheresis is similar to that of conventional apheresis. Briefly, in conventional apheresis blood from a patient in need is pumped extracorporally thru an apheresis device that can remove certain components of the blood using hemofiltration or hemodialysis; and the cleaned blood is returned to the patient. The procedure is described in detail in various publications and in instructions from manufacturers of apheresis equipment and supplies—Haemonetics Corporation (U.S.), Fresenius Medical Care (Germany), Terumo BCT, Inc. (U.S.), Asahi Kasei Medical Co., Ltd. (Japan), Kawasumi Laboratories Inc. (Japan), and others. Note however, that targeted apheresis differs from conventional apheresis in that it selectively binds out specific substances from the blood using immobilized binding agents.

One category of apheresis that is similar in principle to targeted apheresis is therapeutic apheresis. Typically, in therapeutic apheresis the patient's blood is first separated into the cellular fraction (i.e. red blood cells, white blood cells and platelets) and the plasma fraction using centrifugation or membrane filtration. The plasma fraction is then pumped thru an immunoadsorption column consisting of beads coated with a binding agent. The harmful substance in the plasma is bound out and the cleaned plasma is mixed with the blood cellular fraction and returned to the patient. There is however no teaching in conventional therapeutic apheresis of utilizing binding agents that are coated on an apheresis membrane and/or of removing specific harmful substances directly from whole blood.

This invention discloses a novel targeted apheresis method that does not use an immunoadsorbtion column containing coated beads to process the plasma fraction of a patient in need. Instead it teaches a method of treating whole blood using the apheresis membrane as a support matrix on which one or more binding agents and/or ligands are immobilized. When whole blood from a patient in need is passed thru an apheresis device containing these treated membranes the harmful substance in the blood will be bound out and removed from circulation while the cleaned blood is returned to the patient.

In this invention the term “remove” does not mean complete removal of the harmful substance from the blood. It is well known that when the cleaned blood is returned to the patient during therapeutic apheresis there is always a residual level of the harmful substance remaining in the blood of the patient no matter how long or efficiently the apheresis treatment is performed. Generally as a rule of thumb there is approximately a 50% reduction in the level of the substance being removed for every 1.5 times the blood volume of the patient is processed during therapeutic apheresis.

The standard procedure for therapeutic apheresis using a conventional immunoadsorbtion column recommended by most doctors is to limit the procedure to treating 1.5 times the blood volume of the patient. However in the instant invention of targeted apheresis it is preferred that more than 1.5 times the blood volume be processed in order to achieve even more reduction of the harmful substance in the blood. For example, processing 3 times the blood volume would result in removal of 75% of the harmful substance. The lower the concentration of the harmful substance in blood the more likely there will be a better therapeutic response and/or a longer duration of remission. To achieve or maintain a reduced level of the harmful substance in the blood it is often necessary to repeat targeted apheresis one or more times depending on how quickly the concentration of harmful substance in the blood returns to an abnormally high level and the clinical response of the patient.

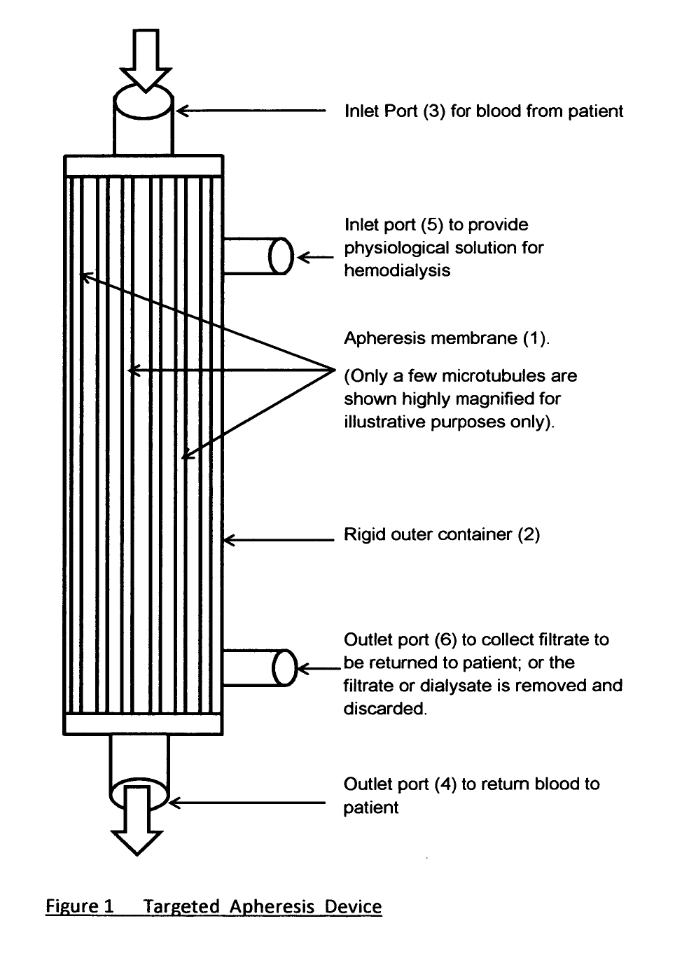

The targeted apheresis device described in this invention utilizes materials and components that are similar to the standard apheresis device used in plasmapheresis. Typically, the targeted apheresis device (FIG. 1) will consist of a semi-permeable membrane configured as a series of stacked hollow fibers or microtubules (1) within a rigid chamber (2). There is an inlet port (3) to allow blood to enter the microtubules; and there is an outlet port (4) to allow the treated blood to return to the patient. Optionally the targeted apheresis device may have two additional ports (5,6) for added versatility. During apheresis the patient's blood is pumped thru the apheresis device utilizing a series of pumps sited before and/or after the apheresis device. As the blood is being pumped thru the device there is positive pressure across the membrane which depending on the porosity of the membrane could cause water and other substances in the blood to filter out. If this feature is not desired during targeted apheresis then there are various methods to eliminate or mitigated this effect. For example, any filtrate collected during targeted apheresis could be mixed with the blood and returned to the patient. Or alternatively the exterior space could be filled with a physiological solution and the ports closed which will limit the amount of material that could filter out. Similarly, the device could be manufactured without the optional ports thus limiting the amount of filtrate that could be lost.

In one embodiment of this invention where targeted apheresis is done in conjunction with hemodialysis and/or hemofiltration two additional ports (5,6) are required. For hemodialysis two ports are required. One inlet port (5) for the physiological solution to enter and one outlet port (6) for removal of the dialysate. For hemofiltration only one outlet (6) is required for removal of the filtrate. In certain instances a combination of both procedures is used. This combination is termed hemodialfiltration.

In one embodiment of this invention the apheresis device is modified and miniaturized into a configuration that is suitable for removing one or more harmful substances that are present at very low concentrations in the blood. i.e. nanogram/ml. or lower. The amount of binding agent to be immobilized on the membrane sufficient to remove these substances is accordingly reduced and consequently the amount of membrane required is also reduced. The final size of the miniaturized targeted apheresis device will be much smaller than a conventional standard apheresis cartridge.

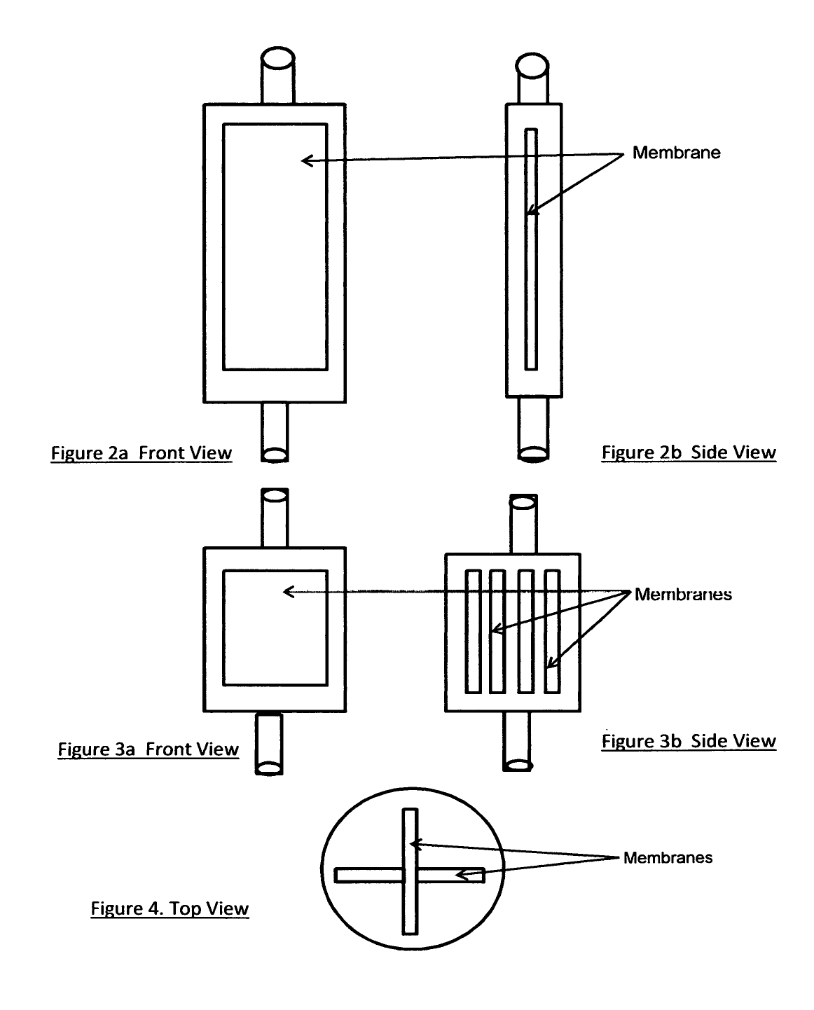

There are many variations on how the membrane is configured in the miniaturized versions. For example the membrane could be a single flat sheet within the apheresis device or a sheet that is folded or pleated (FIG. 2); or it could be multiple sheets that are arranged in parallel (FIG. 3), or in a radial fashion (FIG. 4). It will be obvious to one of skill that there are many other configurations possible without departing from the spirit and scope of this invention. Similarly, there are many different shapes of the outer container from a rectangular chamber with an inlet and an outlet port; to a cylinder with an inlet and an outlet port; and to other shapes that can accommodate the various arrangements of the membrane. The main consideration for these designs is to facilitate the blood flow thru the device as efficiently as possible.

There are important advantages in reducing the area of the membrane and the size of the device. For example, the reduced outer surface area of the membrane and the reduced surface area of the inner walls of the device will result in less exposure to actual contact with whole blood and therefore lower the risk for clot formation and hemolysis. Also the amount of materials and reagents used in manufacturing the device are reduced and the manufacturing process is also made more efficient and cost-effective.

Targeted apheresis can be used to treat to a wide variety of diseases. The following examples are presented to illustrate without limitation how targeted apheresis can be employed in treating different diseases. It will be obvious to one of skill in the art that these methods can be adapted or modified to treat these and other diseases without departing from the spirit and scope of this invention. Said changes are therefore considered to lie within the scope of this invention.

### Example 1

Targeted apheresis to remove infectious pathogens. Patients with a viral infection such as Human Immunodeficiency Virus (HIV) can be treated using targeted apheresis to remove circulating virus. Briefly, binding agents such as anti-HIV antibody or anti-HIV aptamer can be prepared using standard manufacturing techniques and immobilized on the apheresis membrane. When blood from the patient is passed thru the apheresis device the binding agent will bind out the virus and the cleaned blood is returned to the patient. By utilizing immobilized binding agents that target specific pathogens the same procedure can be employed to treat a variety of viral and bacterial diseases including but not limited to HIV, Avian flu and influenza; and also bacteria such as those causing septicemia. Reducing the concentration of pathogens in the blood will alleviate the symptoms of the disease and may potentiate treatment with antibiotics and anti-viral drugs.

### Example 2

Targeted apheresis to treat Rheumatoid Arthritis. Patients with rheumatoid arthritis have rheumatoid factor (RF) and immune complexes in their blood that are responsible for the symptoms of the disease. Rheumatoid factor is an autoantibody to denatured or “altered IgG” and will combine with altered IgG to form immune complexes. By utilizing denatured IgG immobilized on the apheresis membrane it is possible to remove RF and circulating immune complexes. When the blood from a patient with RA is passed thru the apheresis device the RF and immune complexes will bind to altered IgG and be removed from circulation leaving the cleaned blood to be returned to the patient. Reducing the concentration of RF and immune complexes in the blood will alleviate the symptoms of the disease.

### Example 3

Targeted apheresis to treat Autoimmune Disorders. Patients with an autoimmune disease such as Systemic Lupus Erythematosus (SLE), or myasthenia gravis, or multiple sclerosis have elevated levels of autoantibodies in their blood. For example patients with SLE have elevated levels of anti-nuclear antibody (ANA) that can bind to the glomerular basement membrane in the kidney and cause renal failure. Removal of ANA using targeted apheresis may alleviate progression of the disease. To remove circulating ANA the patient's blood is flowed thru an apheresis device in which nuclear antigens are immobilized on the apheresis membrane. The ANA will bind to the antigens and are removed while the cleaned blood is returned to the patient. The same process can be used to remove the autoantibodies associated with different autoimmune diseases by immobilizing the corresponding antigen on the membrane.

### Example 4

Targeted Apheresis to remove pro-inflammatory cytokines. Patients with rheumatoid arthritis and other immune disorders often have elevated levels of pro-inflammatory cytokines such as tumor necrosis factor (TNF) and interleukin 1 (IL-1) in the blood. Removal of these cytokines using targeted apheresis will alleviate the symptoms of the disease. To perform targeted apheresis a binding agent such as an antibody or aptamer to that cytokine is immobilized on the membrane. When blood containing the pro-inflammatory cytokine is flowed thru the device and comes into contact with the binding agent it is bound out and the cleaned blood is returned to the patient. It will be obvious to one of skill in the art that other pro-inflammatory cytokines can be removed in like manner.

### Example 5

Targeted apheresis to treat pre-eclampsia. Pre-eclampsia is a condition that affects some women during pregnancy. It is characterized by high blood pressure, and proteinuria. It is believed that a soluble vascular endothelial growth factor 1 receptor (sVEGFR-1). also known as soluble fat mobilizing like tyrosine kinase 1 receptor (sFlt-1) is responsible for the disease. Removal of sVEGFR-1/sFlt-1 using targeted apheresis would alleviate the symptoms of the disease and prolong pregnancy leading to a healthier baby at birth. To perform targeted apheresis a binding agent that targets sFlt-1 (e.g. anti-sFlt-1 antibody or anti-sFlt-1 aptamer) is immobilized on the apheresis membrane. When blood from the preeclampsia patient is passed thru the targeted apheresis device the binding agent will bind out the sFlt-1 leaving the cleaned blood to be returned to the patient. Reducing the concentration of sFlt-1 in the blood will alleviate the symptoms of preeclampsia. It will be obvious to one of skill in the art that other potentially harmful growth factors in the blood e.g. sEndoglin or Endothelin-1 can be removed in like manner.

### Example 6

Targeted apheresis as an adjunct treatment to cancer therapy using biologics. There are a growing number of biologics being developed to treat cancer. For example, Herceptin® is an antibody that targets the Her2 growth receptor on breast cancer cells; Erbitux® is an antibody that targets the epidermal growth factor receptor on lung cancer cells and Rituxan® is an antibody that targets a cell marker on lymphoma cells. The reason for performing targeted apheresis on cancer patients receiving biologics is that when a cancer cell dies it disintegrates into small fragments some of which form exosomes consisting of cellular material enclosed within parts of the cell membrane. These cellular fragments and exosomes are present in the blood of cancer patients. Therefore when a biologic is injected into a cancer patient some of it will bind to antigens (i.e. growth factor receptors or cell markers) present on the fragments and on the surface of the exosomes. This will reduce the effective concentration of the biologic available to act upon viable cancer cells. This could also cause an immediate adverse reaction of the patient such as fever and chills. Targeted apheresis using immobilized antibodies or binding agent directed against the appropriate growth factor receptor or surface marker can remove these fragments and exosomes thus allowing less adverse reactions when the patient is later injected with the biologic. It will also ensure that more of the biologic is available to treat the cancer.

The above examples illustrate the removal of a specific harmful substance from the blood. Many diseases however, may have multiple harmful substances in the blood. In a further embodiment of this invention it may be desirable to simultaneously remove multiple harmful substances from the blood by utilizing multiple binding agents and/or ligands immobilized upon the apheresis membrane.

### Example 7

Targeted apheresis using multiple binding agents to treat arthritis and other autoimmune diseases. Patients with rheumatoid arthritis and other autoimmune disorders often have elevated levels of pro-inflammatory cytokines such as tumor necrosis factor (TNF) and interleukin 1 (IL-1). Removing pro-inflammatory cytokines in addition to removing RF and immune complexes may provide improved medical benefit to the patient. To perform targeted apheresis multiple binding agents and/or ligands (i.e. altered IgG, anti-TNF antibody, and anti-IL-1 antibody) are immobilized upon the membrane. When blood from a patient with an autoimmune disease is passed thru the targeted apheresis device RF, Immune complexes, TNF and IL-1 are removed and the cleaned blood returned to the patient. Reducing the concentration of RF, immune complexes, TNF and IL-1 in the blood will alleviate the symptoms of the disease. It will be obvious to one of skill in the art that other binding agents such as aptamers and binding peptides can be substituted in lieu of antibody against one or more of the targets. It will also be obvious to one of skill in the art that other pro-inflammatory substances in the blood can be removed in like manner.

### Example 8

Targeted apheresis using multiple binding agents to treat preeclampsia. Women with preeclampsia have elevated levels of multiple biomarkers in their blood including sFlt-1, soluble Endoglin and proinflammatory cytokines such as TNF and IL-1. Removing these factors using targeted apheresis could alleviate the symptoms of preeclampsia and prolong pregnancy. To perform targeted apheresis multiple binding agents and/or ligands (i.e. anti-sFlt-1 antibody, anti-endoglin antibody, anti-TNF antibody and anti-II-1 antibody) are immobilized upon the membrane. When blood from a patient with preeclampsia is passed thru the targeted apheresis device sFlt-1, Endoglin, TNF and IL-1 are removed and the cleaned blood returned to the patient. Reducing the concentration of these biomarkers in the blood will alleviate the symptoms of preeclampsia. It will be obvious to one of skill in the art that other binding agents such as aptamers and binding peptides can be substituted in lieu of antibody against one or more of the targets. It will also be obvious to those of skill in the art that other harmful biomarkers involved in pre-eclampsia can be removed in like manner.

Although targeted apheresis is designed to specifically remove one or more harmful substances from the blood there are instances where it would be desirable to combine it with hemofiltration and/or hemodialysis in order to provide additional therapeutic benefit to the patient.

### Example 9

Targeted apheresis with hemofiltration and/or hemodialysis. In certain circumstances it may be desirable to combine targeted apheresis with hemofiltration and/or hemodialysis. For example women with preeclampsia have high blood pressure with edema and proteinuria. Therefore in combination with targeted apheresis removing excess water and/or toxic waste material from the blood using hemofiltration and/or hemodialysis could be of additional benefit in treating this condition. In these instances the targeted apheresis device would have two additional ports to allow dialyzing solution to enter and/or the dialysate and filtrate to exit.

To combine hemofiltration with targeted apheresis a positive pressure differential is applied across the membrane. Depending on the pore size of the membrane employed water, electrolytes and small molecules will filter thru the membrane and can be removed. Reducing excess fluid from the circulation could reduce high blood pressure and also reduce stress on kidney function. In this instance only one additional port to remove the filtrate is required.

To combine hemodialysis with targeted apheresis a physiological solution is flowed exterior to the semi-permeable membrane which will allow toxic material in the blood to dialyze out and be removed. Removing toxic waste material could reduce stress on kidney function and alleviate the symptoms of disease. In this instance two additional ports are required. One port for the dialyzing solution to enter and one port for the dialysate to be removed.

This invention teaches the removal of harmful substances from whole blood. It will be obvious to one of skill in the art that the same methods can be applied to remove harmful substances from the plasma fraction without departing from the spirit and scope of this invention. For example, in one embodiment of this invention the plasma faction is separated from the cellular blood elements using differential centrifugation or membrane filtration. The plasma fraction is then treated using targeted apheresis in the same manner that was used to treat whole blood. The cleaned plasma is then mixed with the cellular blood fraction and the reconstituted whole blood returned to the patient.

In one embodiment of this invention the targeting apheresis device is regenerated after each use so that it can be used multiple times. Briefly, an eluting solution such as a glycine-HCl buffer pH 2.8 is pumped thru the device causing any bound material to elute off the membrane and be discarded. A physiological solution such as phosphate buffered saline pH 7.4 is then pumped thru the device to restore the binding capacity of the binding agents. The device is then stored at 4 degree C. until it is next required. Or depending on the stability of the binding agent the membrane could be washed with distilled water, dried and stored under vacuum. It will be obvious to one of skill in the art that there are other eluting agents that can be used, and there are also other methods of preserving and storing the device. These and other methods of regenerating devices containing biological material are considered to lie within the scope of this invention.

In one embodiment of this invention where a large amount of circulating harmful substances is to be removed the targeted apheresis device is periodically regenerated and reused during apheresis. Briefly, two targeting apheresis devices are attached in parallel to the blood catheter from the patient. The blood is alternatively treated using one device while the other device is being regenerated. The process of regeneration is similar to that described earlier where any bound material is eluted off the membrane and discarded; and the device treated to restore its binding capacity. The method of alternatively utilizing one apheresis device while the other is being regenerated is well known to those of skill in the art and described by several manufacturers of apheresis equipment and devices (e.g. Liposorber®, Kaneka Pharma America; Therasorb® Miltenyi Biotec).

This invention teaches a novel means of performing targeted apheresis that can be employed to treat a variety of diseases. The list of diseases that can be treated using targeted apheresis are not limited to those examples disclosed in this invention. These examples are provided to illustrate the versatility of this method of targeted apheresis and how it could be applied to many different types of diseases.

From the teachings in this invention it will be obvious to one of skill in the art to be able to adapt, modify or otherwise change certain aspects of the materials and methods described herein without departing from the spirit and scope of this invention. Such changes therefore are considered to lie within the scope of this invention.

What is claimed is:

1. A method of targeted apheresis utilizing one or more binding agents and/or ligands immobilized on an apheresis membrane enclosed within an apheresis device.

2. According to claim 1 the binding agents includes antibodies, aptamers, binding peptides and cellular receptors.

3. According to claim 1 the chemical composition of the apheresis membrane is cellulose, or cellulose diacetate; or polypropylene; or polysulfone; or polymethylmethacrylate or like materials; and any combination of these materials.

4. According to claim 1 the apheresis membrane used includes semi-permeable membranes, permeable membranes and impermeable membranes.

5. According to claim 1 the method of immobilizing the binding agent or ligand includes absorption or covalent binding of the binding agent to the apheresis membrane either directly or indirectly thru a linking molecule.

6. According to claim 1 whole blood from a patient in need is passed thru a targeted apheresis device whereby harmful substances in the blood are removed and the cleaned blood returned to the patient.

7. According to claim 1 plasma from a patient in need is passed thru a targeted apheresis device whereby harmful substances in the plasma are removed and the cleaned plasma is mixed with the cellular blood elements and returned to the patient.

8. According to claim 1 a method of treating infectious diseases using targeted apheresis.

9. According to claim 1 a method of treating autoimmune disorders using targeted apheresis

10. According to claim 1 a method of treating preeclampsia using targeted apheresis.

11. According to claim 1 a method of removing interfering substances such as cell fragments and exosomes from the blood of cancer patients using targeted apheresis, prior to the patient later receiving immunotherapy.

12. A method of combining hemofiltration and/or hemodialysis with targeted apheresis to treat a patient in need.
